(19) United States
(12) Patent Application Publication
(10) Pub. No.: US 2025/0009519 A1
(43) Pub. Date: Jan. 9, 2025

(54) METHOD FOR PRODUCING A MEDICAL PRODUCT, AND CORRESPONDING MEDICAL PRODUCT

(71) Applicant: Aesculap AG, Tuttlingen (DE)

(72) Inventors: Josef Hauser, Gunningen (DE); Georg Hettich, Freiburg (DE); Tobias Waeschle, Koenigsheim (DE); Frank Fedtke, Emmingen (DE); Johannes Adam, Tuttllingen (DE); Steffen Schaz, Neuhausen (DE)

(21) Appl. No.: 18/709,942

(22) PCT Filed: Nov. 25, 2022
(86) PCT No.: PCT/EP2022/083263

§ 371 Date: May 14, 2024

(30) Foreign Application Priority Data
Nov. 26, 2021 (DE) 10 2021 213 351.7

(51) Int. Cl. A61F 2/30; B33Y 10/00

(52) U.S. Cl. CPC A61F 2/3094; B33Y 10/00; A61F 2002/30968; A61F 2002/30985

(57) ABSTRACT

A method for producing a medical product includes the step of forming at least one functional element on a surface of a base body by additive construction or manufacture, followed by the step of connecting the at least one functional element to the base body.

### CROSS-REFERENCE TO RELATED APPLICATIONS

This application is the United States national stage entry of International Application No. PCT/EP2022/083263, filed on Nov. 25, 2022, and claims priority to German Application No. 10 2021 213 351.7, filed on Nov. 26, 2021. The contents of International Application No. PCT/EP2022/083263 and German Application No. 10 2021 213 351.7 are incorporated by reference herein in their entireties.

### FIELD

The invention relates to a method for producing a medical product and to a medical product.

### BACKGROUND

The use of hip joint and knee joint total endoprostheses is among the most successful of surgical treatment options. Notwithstanding, many revision operations are required every year.

One possible way of increasing the lifetime of a hip or knee joint implant, and therefore of reducing the number of revision operations, consists in the provision of patient-specific, that is to say personalized, implants, which are produced by means of additive manufacturing. A disadvantage of implants configured in this way is, however, the long time between the decision by a surgeon to use such an implant and its clinical availability. In order to shorten this time, in current provision approaches work is already being carried out with 3D printers in the clinical field. This form of implant provision is not, however, applicable to implants for which finishing is required, for example milling of interfaces or polishing of glide faces, in order to achieve the accuracy and/or surface quality that are required from a medical viewpoint. This is true particularly for hip or knee joint implants, but also for other medical products, for example surgical instruments.

### SUMMARY

It is an object of the invention to provide a method for producing a medical product and a medical product, which partially or fully avoid the disadvantages occurring in the prior art, in particular as described above.

According to a first aspect, the invention relates to a method for producing a medical product, in particular an implant, preferably a hip joint or knee joint implant (hip joint or knee joint endoprosthesis) or a surgical instrument.

Preferably, the medical product is an endoprosthesis, in particular a total endoprosthesis, a hemi-endoprosthesis or a partial endoprosthesis. The endoprosthesis may in particular be selected from the group consisting of a hip joint endoprosthesis, a knee joint endoprosthesis, a shoulder joint endoprosthesis, an elbow joint endoprosthesis, an ankle joint endoprosthesis and a finger joint endoprosthesis.

Alternatively, the medical product may preferably be a medical, in particular surgical, instrument. The medical instrument may in particular be selected from the group consisting of a dilation instrument (dilating instrument), a grip instrument (gripping instrument), retention and/or a clamp instrument (holding and/or clamping instrument), and a section and/or separation instrument (cutting and/or separating instrument). The dilation instrument may in particular be selected from the group comprising a wound hook, retractor, spreaders and specula. The grip instrument may in particular be selected from the group comprising tweezers, a clamp, forceps and needle holders. The retention and clamp instrument may in particular be selected from the group comprising a clamp, such as a clamp for temporary clamping for the intestine and/or blood vessels and a preparatory clamp. The section and/or separation instrument may in particular be selected from the group comprising a blade (scalpel) and scissors.

The method comprises the following step:
a) additive or generative construction or additive or generative manufacture, that is to say additive or generative production, of at least one functional element on a surface of a base body,
wherein the at least one functional element is connected, in particular indirectly or directly, to the base body after step a) is carried out.

Preferably, the at least one functional element is connected, in particular indirectly or directly, to the base body by substance or material bonding after step a) is carried out.

The term “functional element” is intended in the context of the present invention to mean a part or subsection of a whole, a function being assigned to the part or subsection.

The term “at least one functional element” is intended in the context of the present invention to mean a functional element, that is to say only one functional element, or a multiplicity of functional elements, that is to say two or more functional elements.

The term “base body” is intended in the context of the present invention to mean an individual part or an individual component, in particular a prefabricated individual part (so-called prefab part) or a prefabricated component (so-called prefab component), to which in general a function is (likewise) assigned.

The term “component” is intended in the context of the present invention to mean an individual or prefab part, in particular a nondestructively dividable individual or prefab part.

The term “group of components” is intended in the context of the present invention to mean an arrangement or a corresponding complex consisting or composed of two or more components, and in particular an arrangement or a corresponding complex produced by an assembly process.

The term “endoprosthesis” is intended in the context of the present invention to mean an artificial joint, that is to say a joint replacement.

The term “total endoprosthesis” is intended in the context of the present invention to mean an artificial joint replacement that is configured for the replacement of an entire natural joint, that is to say a natural joint head and a natural joint socket.

The term “hemi-endoprosthesis” is intended in the context of the present invention to mean an artificial joint replacement that is configured only to replace one half of a natural joint, for example only a natural joint head or only a natural joint socket.

The term “partial endoprosthesis” is intended in the context of the present invention to mean an artificial joint replacement that is configured only to replace a part of a natural joint.

By the invention, the advantages of conventional, that is to say non-additive or non-generative manufacture, for example high accuracy and surface finish, may be realized together with the advantages of additive or generative manufacture, for example patient-specific or personalized adaptation or alignment of the medical product, provision of the medical product “on site” and real-time production, and therefore manufacturing on demand. By the invention, a so-called monobloc medical product, that is to say a medical product formed in one part, is preferably provided.

In an embodiment of the invention, the base body is produced by means of a non-additive or non-generative manufacturing method, in particular selected from the group consisting of primary shaping methods, reshaping methods, separating methods, joining methods, coating methods and material property modification methods, and combinations of at least two of the aforementioned manufacturing methods. In other words, the base body is preferably not produced by additive or generative manufacturing methods. In this way, properties that can be achieved to a sufficient extent only by conventional manufacturing methods, for example high accuracy and surface quality, may advantageously be realized. Preferably, the base body is produced by means of a method that is selected from the group consisting of casting, forging, rolling, extrusion, deep drawing, bending, sawing, planing, milling, boring, stamping, cutting, for instance shear cutting and/or flame cutting, electrical discharge machining, welding, soldering, riveting, screwing, assembling, adhesive bonding, varnishing, electroplating, powder coating, hot-dip galvanizing, hardening, annealing, polishing, for instance electropolishing, and combinations of at least two of the aforementioned (non-additive or non-generative) manufacturing methods. According to the invention, it is therefore preferred in particular for the at least one functional element to be manufactured or produced by means of an additive or generative manufacturing method and for the base body to be produced by means of a non-additive or non-generative manufacturing method. To this extent, the medical product in the context of the present invention may be referred to as a hybrid medical product, in particular as a hybrid implant, preferably a hybrid endoprosthesis, or medical, in particular surgical, hybrid instrument.

In principle, the same material, in particular the same substance, may be used for the additive construction or manufacture of the at least one functional element as for the production of the base body. According to the invention, it may however be preferred in particular for a different material, in particular a different substance, to be used for the additive construction or manufacture of the at least one functional element than for the production of the base body.

The material for the additive construction or manufacture of the at least one functional element and the material for the production of the base body may be selected independently of one another from the group consisting of metal, alloy, hard metal, ceramic, plastic and combinations, in particular mixtures, of at least two of the aforementioned materials.

The metal may in particular be selected from the group consisting of titanium, cobalt, chromium, iron, magnesium, zinc, niobium, tantalum, zirconium and combinations, in particular alloys, of at least two of the aforementioned metals.

The alloy may in particular be selected from the group consisting of iron alloy, cobalt alloy, chromium alloy, niobium alloy, zirconium alloy, alloys containing cobalt and chromium, chromium-cobalt-molybdenum alloy and combinations, in particular mixtures, of at least two of the aforementioned alloys.

The iron alloy may in particular be steel, preferably stainless steel, in particular corrosion-resistant stainless steel.

The chromium-cobalt-molybdenum alloy may in particular have a proportion of from 62 wt % to 66 wt % cobalt, from 27 wt % to 31 wt % chromium and from 4 wt % to 5 wt % molybdenum, in each case expressed in terms of the total weight of the alloy.

The ceramic may in principle be a non-oxide ceramic, an oxide ceramic or a combination thereof. The non-oxide ceramic may in particular be selected from the group consisting of chromium nitride (CrN), chromium carbonitride (CrCN), chromium zirconium nitride (CrZrN), zirconium nitride (ZrN) and combinations of at least two of the aforementioned non-oxide ceramics. The oxide ceramic may in particular be selected from the group consisting of aluminum oxide (Al₂O₃), zirconium dioxide (ZrO₂), titanium dioxide (TiO₂), chromium(III) oxide (Cr₂O₃) and combinations of at least two of the aforementioned oxide ceramics. The aforementioned aluminum oxide is preferably an aluminum oxide of the corundum type. The aforementioned zirconium dioxide is preferably a zirconium dioxide of the baddeleyite type. The aforementioned titanium dioxide is preferably a titanium dioxide of the rutile type. The aforementioned chromium(III) oxide is preferably a chromium(III) oxide of the corundum type.

The term “hard metal” is intended in the context of the present invention to mean a metal-matrix composite in which the hard substances, which are present as particles, in particular small particles, are held together by a matrix of a metal or an alloy. Hard metals are therefore somewhat less hard than the pure hard substances, but are significantly tougher. On the other hand, they are harder than pure metals, alloys and hardened steel. Preferably, the hard metal comprises a metal matrix of cobalt and/or nickel and a hard substance. The hard substance is preferably selected from the group consisting of tungsten carbide (WC), titanium carbide (TiC), titanium nitride (TiN), niobium carbide, tantalum carbide, vanadium carbide and mixtures of at least two of the aforementioned hard substances. In particular, the hard metal may have a nickel proportion of from 8 wt % to 20 wt % and a metal carbide proportion, in particular a tungsten carbide proportion, of from 80 wt % to 92 wt %, expressed in terms of the total weight of the hard metal.

The plastic may in principle be an in vivo degradable or in vivo resorbable plastic and/or an in vivo non-degradable or in vivo non-resorbable plastic.

The plastic may in particular be selected from the group consisting of polyethylene, polypropylene, polyethylene terephthalate, polypropylene terephthalate, polybutylene terephthalate, polyamide, polyurethane, polyglycolic acid or polyglycolide, polylactic acid or polylactide, poly-3-hydroxybutyric acid or poly-3-hydroxybutyrate, poly-4-hydroxybutyric acid or poly-4-hydroxybutyrate, polytrimethylene carbonate, poly-para-dioxanone, poly-8-caprolactone and mixtures, that is to say blends, of at least two of the aforementioned plastics.

The polyethylene may in particular be high density polyethylene (HDPE), low density polyethylene (LDPE), high molecular weight polyethylene (HMWPE), ultrahigh molecular weight polyethylene (UHMWPE), copolymers of at least two of the aforementioned types of polyethylene or mixtures, that is to say blends, of at least two of the aforementioned types of polyethylene.

The term “additive manufacturing method” or “generative manufacturing method” is intended in the context of the present invention to mean manufacturing methods in which a material or substance, in particular a metal, a hard metal, an alloy, a ceramic, a plastic or a combination of at least two of the aforementioned materials or substances, is applied layer by layer and three-dimensional objects are thus generated. Preferably, the layerwise construction is carried out under computer control from one or more liquid or solid materials or substances according to predetermined dimensions and shapes (CAD and/or CAM). During the construction, physical and/or chemical hardening or melting processes generally take place. Typical materials or substances are metals, alloys, plastics, synthetic resins and ceramics.

In a further embodiment of the invention, step a) is carried out by means of deposit welding, in particular laser deposit welding, preferably laser powder deposit welding or laser wire deposit welding.

In laser deposit welding, surface deposition is carried out onto a workpiece or component by means of melting and simultaneous application of a material. The material may be carried out in powder form, for example as a metal powder (laser powder deposit welding) or in the form of a welding wire or tape (laser wire deposit welding). A laser, in particular a high-power laser, is used as the heat source. The laser may in particular be a diode laser, fiber laser, CO₂ laser or Nd:YAG laser. The term “diode laser” is intended in the context of the present invention to mean a laser, the light of which is generated with laser diodes, that is to say with semiconductor materials. Diode lasers can generate light powers of up to more than 60 kW. The term “fiber laser” is intended in the context of the present invention to mean a solid-state laser, the active medium of which is formed by a doped core of a glass fiber. It is therefore a glass laser having optical waveguide properties. The laser radiation that is guided by the laser-active fiber experiences a very high amplification because of the long length. A fiber laser has one or more dot laser diodes, input coupling optics (discretely or fiber-coupled diode laser spliced onto the cladding) and a resonator. The fiber typically consists of a plurality of layers. The main part is usually of quartz glass, for example 0.25 mm thick, enclosed by a thin layer of plastic. The active core is much thinner, for example 10 μm, and predominantly consists of doped quartz glass, for example with a low percentage of aluminum and a few parts per thousand of rare earths. Fiber lasers can have electro-optical efficiencies of up to more than 30%. The term “carbon dioxide laser” or “CO₂ laser” is intended in the context of the present invention to mean a laser, the laser medium of which comprises a CO₂—N₂—He (helium) gas mixture or consists of such a gas mixture. The N₂ molecules are excited in a resonator by a DC or RF glow discharge. CO₂ lasers can achieve output powers of up to 80 kW and pulse energies of up to 100 kJ. A CO₂ laser produces a beam of infrared light with a wavelength in the bands of 9.4 μm and 10.6 μm. The efficiency is about 15% to 20%. The term “Nd:YAG laser” is intended in the context of the present invention to mean a solid-state laser (neodymium-doped yttrium aluminum garnet laser), which uses a neodymium-doped YAG crystal as the active medium and usually emits infrared radiation with a wavelength of 1064 nm. Further transitions exist at 946 nm, 1320 nm and 1444 nm.

Laser deposit welding may be used to generate both layers and freely formed 2.5D structures. In the latter case, it may be categorized among 3D printing methods. During laser deposit welding with a powder, the laser heats the workpiece or component, usually in a defocused fashion, and melts it locally. At the same time, an inert gas mixed with a fine metal powder is supplied. The supply of the active region with the metal/gas mixture is preferably carried out by means of nozzles, for example trailing or coaxial nozzles. At the heated location, the metal powder melts and bonds with the material, in particular metal, of the workpiece or component. Besides metal powders, ceramic powder substances, especially hard substances, may also be used. Laser deposit welding with a wire or tape works in a similar way to the method with powders, but with a wire or tape as the filler substance.

In a further embodiment of the invention, step a) is carried out by means of beam sintering, in particular laser sintering, preferably selective laser sintering. Laser sintering is a generative layer construction method, in which a workpiece or component is constructed layer by layer. By the effect of the laser beams, arbitrary three-dimensional geometries, in particular ones having undercuts, may be generated. In this way, in particular, it is possible to produce workpieces or components that cannot be produced with the aid of conventional mechanical or casting technology manufacturing methods. Usually, a CO₂ laser, an Nd:YAG laser or a fiber laser is employed as the laser. The substance used for producing the workpiece or component is preferably in powder form. In particular, a plastic powder, plastic-coated molding sand, a metal or alloy powder or a ceramic powder can be used. Preferably, the powder is applied to the workpiece or component surface-wide, particularly in a thickness of from 1 μm to 200 μm, with the aid of a blade or roller. The layers are then sintered or melted stepwise on the surface of the workpiece or component by driving the laser beam according to the layer contour of the workpiece or component. The workpiece or component is then lowered slightly and a new layer is applied. The processing is carried out step by step in the vertical direction. It is thereby also possible to generate undercut contours. The energy that is supplied by the laser is absorbed by the substance, which is preferably in powder form, and leads to locally limited sintering of particles with a reduction of the overall surface area.

In a further embodiment of the invention, step a) is carried out by means of beam melting, in particular laser melting, preferably selective laser melting. In laser melting, the substance to be processed is applied in powder form as a thin layer onto a workpiece or component. The material in powder form is locally fused fully by means of laser radiation and forms a solid material layer after solidification. The workpiece or component is subsequently lowered by the amount of one layer thickness and powder is again applied. This cycle is repeated until all the layers have been fused. Excess powder is cleaned off from the finished workpiece or component, which according to requirements is processed or used directly. The typical layer thicknesses for the construction of the workpiece or component are preferably between 15 μm and 500 μm for all materials. The data for the guiding of the laser beam are generated by means of software from a 3D CAD body. In a first calculation step, the workpiece or component is subdivided into individual layers. In a second calculation step, the paths (vectors) that the laser beam follows are generated for each layer. In order to avoid contamination of the substance with oxygen, the process may advantageously be carried out in a protective gas atmosphere, for example with argon and/or nitrogen.

Alternatively, step a) may be carried out by means of other additive or generative manufacturing methods, for example by means of electron beam melting, in particular selective electron beam melting (electron beam sintering). In this case a substance in powder form, preferably metal powder, is melted in a controlled way with the aid of an electron beam as energy source, so that in particular compact components with almost arbitrary geometry can be produced directly from design data. On the basis of a digital 3D model, for this purpose a layer of powder, in particular metal powder, is alternately applied surface-wide onto a component with a blade and initially preheated over a large area by means of an electron beam, and subsequently locally melted. After cooling, the melt solidifies to form a solid layer, in particular a metal layer, with an approximately 100% structural density. The component is then lowered by one layer thickness and a subsequent layer of powder, in particular metal powder, is applied onto the previous layer. These steps are repeated many times. Preferably, after the manufacturing process per se, loose powder is removed from the component with compressed air. In this way, the desired component is generated layerwise. In order to prevent a reaction from taking place with ambient gases, and the substance in powder form thereby being modified in its material properties, the process is preferably carried out at a reduced pressure or in a vacuum.

In a further embodiment of the invention, the at least one functional element is configured differently than the base body when carrying out step a). In particular, step a) may be carried out in such a way that the at least one functional element is distinguishable from the base body in relation to at least one feature, the at least one feature preferably being selected from the group consisting of shape, material, hardness, flexibility, dimensions, in particular height and/or depth and/or length and/or diameter, and combinations of at least two of the aforementioned features.

Preferably, a component, a component section or a group of components of the medical product is used as the base body.

Preferably, a component, a component section or a group of components of the medical product is additively constructed or manufactured as functional element when carrying out step a).

In a further embodiment of the invention, a component, a component section or a group of components of an implant, preferably of an endoprosthesis, in particular a total endoprosthesis, a hemi-endoprosthesis or a partial endoprosthesis, is used as the base body.

In a further embodiment of the invention, when carrying out step a) a component, a component section or a group of components of an implant, preferably of an endoprosthesis, in particular a total endoprosthesis, a hemi-endoprosthesis or a partial endoprosthesis, is additively constructed or manufactured on the surface of the base body as at least one functional element.

In a further embodiment of the invention, the endoprosthesis is selected from the group consisting of a hip joint endoprosthesis, a knee joint endoprosthesis, a shoulder joint endoprosthesis, an elbow joint endoprosthesis, an ankle joint endoprosthesis and a finger joint endoprosthesis. The knee joint endoprosthesis may, in particular, be a unicondylar or bicondylar knee joint endoprosthesis.

Preferably, the endoprosthesis is a hip joint endoprosthesis, in particular a hip joint total endoprosthesis, a hip joint hemi-endoprosthesis or a hip joint partial endoprosthesis, or a knee joint endoprosthesis, in particular a knee joint total endoprosthesis, a knee joint hemi-endoprosthesis or a knee joint partial endoprosthesis.

Preferably, the component or the group of components of the implant is selected from the group consisting of an artificial joint socket, an artificial joint socket insert, an artificial joint head insert, an artificial joint head, joint shaft, cylinder or cone for a joint shaft, and groups of components consisting of at least two of the aforementioned components.

In a further embodiment of the invention, the component or the group of components of the implant is selected from the group consisting of an artificial acetabulum, an artificial acetabular insert, an artificial femoral head insert, an artificial femoral head, a hip joint shaft, a cylinder or cone for a hip joint shaft, and groups of components consisting of at least two of the aforementioned components.

In a further embodiment of the invention, the component or the group of components of the implant is selected from the group consisting of an upper leg prosthesis (so-called femoral component), a shaft for an upper leg prosthesis, a lower leg prosthesis (so-called tibial component), a shaft for a lower leg prosthesis, and groups of components consisting of at least two of the aforementioned components.

In a further embodiment of the invention, a component that is cylindrical or conical at least in sections, in particular a component, in particular prefab part, that is cylindrical or conical only in sections, or a component that is cylindrical or conical continuously (in the longitudinal direction) is used as the base body, and when carrying out step a) a shaft, in particular a joint shaft, preferably a hip joint shaft, is additively constructed or manufactured as at least one functional element on a surface of the component, in particular prefab part, that is cylindrical or conical at least in sections.

In a further embodiment of the invention, an upper leg prosthesis (femoral component) or lower leg prosthesis (tibial component) is used as the base body, and when carrying out step a) a shaft, in particular an upper leg or lower leg shaft, is additively constructed or manufactured as at least one functional element on a surface of the upper or lower leg prosthesis.

In a further embodiment of the invention, a component, a component section or a group of components of a medical, in particular surgical, instrument is used as the base body.

In a further embodiment of the invention, when carrying out step a) a component, a component section or a group of components of a medical, in particular surgical, instrument is constructed or manufactured as at least one functional element on the surface of the base body.

In a further embodiment of the invention, the medical, in particular surgical, instrument is selected from the group consisting of a dilation instrument (dilating instrument), a grip instrument (gripping instrument), retention and/or a clamp instrument (holding and/or clamping instrument), and a section and/or separation instrument (cutting and/or separating instrument). The dilation instrument may in particular be selected from the group comprising a wound hook, retractor, spreaders and specula. The grip instrument may in particular be selected from the group comprising tweezers, a clamp, forceps and needle holders. The retention and clamp instrument may in particular be selected from the group comprising a clamp, such as a clamp for temporary clamping for the intestine and/or blood vessels and a preparatory clamp. The section and/or separation instrument may in particular be selected from the group comprising a blade (scalpel) and scissors.

In a further embodiment of the invention, the component, the component section or the group of components of the medical, in particular surgical, instrument is selected from the group consisting of branches or branch sections (arm parts or arm sections), grips or grip sections, a link or link section, mouth parts or mouth sections, gripping or retaining jaws or gripping or retaining jaw sections, holding and/or clamping jaws or holding and/or clamping jaw sections, cutting and/or separating jaws or cutting and/or separating jaw sections, and groups of components consisting of at least two of the aforementioned components.

In a further embodiment of the invention, a group of components of a medical, in particular surgical, instrument is used as the base body, the group of components comprising branches or branch sections mounted movably in rotation or pivoting, in particular shearing, on one another by means of a joint or connection, in particular a link, each branch or each branch section comprising a grip section at a proximal end, and when carrying out step a) a functional section, in particular selected from the group consisting of a gripping or retaining jaw or gripping or retaining jaw section, a holding and/or clamping jaw or holding and/or clamping jaw section, and a cutting and/or separating jaw or cutting and/or separating jaw section, respectively is additively constructed or manufactured as at least one functional element on a distal end of the branches or of the branch sections.

The term “link” is intended in the context of the present invention to mean a connection, particularly in the form of a joint, by means of which two branches of a medical, in particular surgical, instrument are mounted movably in rotation or pivoting, in particular shearing, on one another.

In a further embodiment of the invention, a group of components of a medical, in particular surgical, instrument is used as the base body, the group of components comprising two branches or branch sections mounted movably in rotation or pivoting, in particular shearing, on one another by means of a joint or connection, in particular a link, each branch or each branch section comprising a grip or grip section at a proximal end and a functional section at a distal end, in particular selected from the group consisting of a gripping or retaining jaw or gripping or retaining jaw section, a holding and/or clamping jaw or holding and/or clamping jaw section, and a cutting and/or separating jaw or cutting and/or separating jaw section, each functional section having a recess, in particular an elongate recess, for receiving an insert, and when carrying out step a) an insert, in particular a recess shape-complementary insert, that is to say a recess that is complementary in shape to the shape of the respective recess, is additively constructed or manufactured respectively as at least one functional element in the recesses. The insert is preferably a metal insert, that is to say an insert that comprises a metal or consists of a metal, in particular a hard metal insert, that is to say an insert that comprises a hard metal or consists of a hard metal. In respect of suitable metals, in particular hard metals, reference is made to the description above.

Preferably, chronologically following step a), the method furthermore comprises a step b) of thermal aftertreatment, in particular annealing. Preferably, step b) is carried out by means of a laser beam. By the thermal aftertreatment step, with particular advantage, possible distortions or stresses occurring in the base body and/or the at least one functional element when carrying out step a) can be eliminated or at least reduced.

According to a second aspect, the invention relates to a medical product, in particular an implant, preferably an endoprosthesis, in particular a hip joint or knee joint endoprosthesis, or a medical, in particular surgical, instrument.

The medical product comprises at least one functional element produced by means of an additive or generative manufacturing method and a base body.

The base body is preferably not produced by means of an additive or generative manufacturing method. In other words, the base body is preferably produced by means of a non-additive or non-generative manufacturing method.

The at least one functional element is connected to the base body, in particular indirectly or directly. Preferably, the at least one functional element is connected to the base body by substance or material bonding.

Alternatively or in combination, the medical product is produced or producible by a method according to the first aspect of the invention.

In respect of further features and advantages of the medical product, reference is fully made to the comments given in the scope of the first aspect of the invention. The features and advantages described there in relation to the method and the medical product also apply correspondingly for the medical product according to the second aspect of the invention.

Further features and advantages of the invention may be found in the following description of preferred embodiments of the invention, which are explained with the aid of figures and associated figure descriptions. Individual features of the invention may in this case respectively be implemented independently or in combination with one another. The exemplary embodiments described serve merely for further explanation of the invention, without the latter being restricted thereto.

### BRIEF DESCRIPTION OF THE DRAWINGS

The following is schematically shown in the figures:

FIG. 1 shows the conduct of a laser deposit welding method according to the invention;

FIG. 2 shows an embodiment of a medical product according to the invention;

FIG. 3 shows a further embodiment of a medical product according to the invention;

FIG. 4 shows a further embodiment of a medical product according to the invention;

FIG. 5 shows a further embodiment of a medical product according to the invention;

FIG. 6 shows a further embodiment of a medical product according to the invention; and

FIG. 7 shows a further embodiment of a medical product according to the invention.

### DETAILED DESCRIPTION

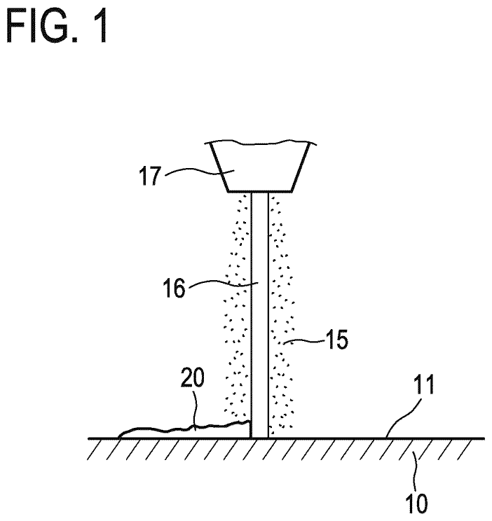

FIG. 1 schematically shows an embodiment of a method according to the invention.

For the additive construction or manufacture of a functional element 20, a material 15 in powder form is applied on a surface 11 of a base body 10 by means of laser powder deposit welding. For this purpose, the material 15 is heated with the aid of a laser beam 16, particularly in a defocused fashion, and melted on the surface 11 of the base body 10. The application of the material 15 in powder form onto the surface 11 of the base body 10 is preferably carried out by means of a nozzle 17. The nozzle 17 may for example be configured as a coaxial, multijet or wide-jet nozzle. Preferably, the material 15 in powder form is applied continuously onto the surface 11 of the base body 10. The material 15 in powder form is heated in the laser beam and subsequently melted in a construction and joining region on the surface 11 of the base body 10. For example a metal, an alloy or a ceramic may be used as the material 15 in powder form.

Laser powder deposit welding advantageously allows a high precision of the material application, and in particular deposit welding in a plurality of layers. Metallurgical fusion of the applied layers takes place, and therefore a connection to the base body 10 by substance or material bonding. Because of the low energy input in comparison with conventional welding methods, both the additively constructed or manufactured functional element 20 and the base body 10 experience less distortion, and in particular no structural damage.

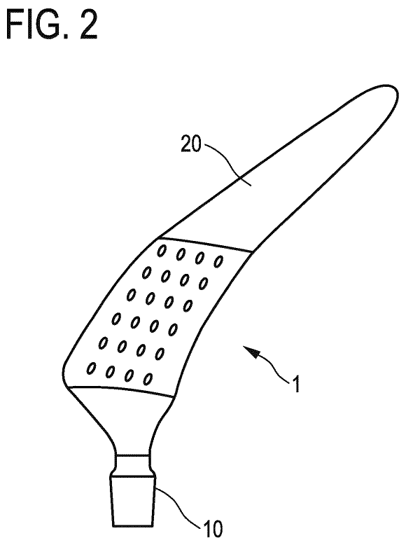

FIG. 2 schematically shows an embodiment of a medical product 1 according to the invention in the form of a hip implant 1. The hip implant 1 comprises a conically configured base body 10 and a functional element 20 in the form of a shaft. The functional element 20 is connected to the base body 10, preferably by substance or material bonding. In order to produce the hip implant 1, the base body 10 is preferably used as a prefabricated, in particular ground and/or polished component, and the functional element 20 is additively constructed or manufactured onto one of its end faces, preferably by means of laser deposit welding, selective laser melting or selective laser sintering. The base body 10 is preferably produced with the aid of a non-additive manufacturing method.

In respect of suitable materials for the base body 10 and the functional element 20, reference is made to the materials disclosed in the general, or preceding, description for the base body and the at least one functional element.

The hip implant shown in FIG. 2 may also be referred to as a hybrid implant in the context of the present invention.

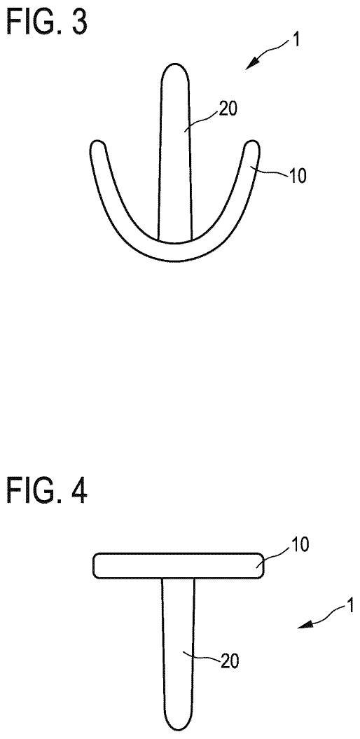

FIG. 3 schematically shows a further embodiment of a medical product 1 according to the invention in the form of a knee partial endoprosthesis 1.

The knee partial endoprosthesis 1 comprises a base body in the form of a femoral component 10 and a functional element 20 connected, in particular connected by substance or material bonding, to the femoral component 10. The functional element 20 is preferably configured in the form of a shaft.

In order to produce the knee partial endoprosthesis 1, the femoral component 10 is preferably used as a prefabricated component, that is to say as a prefab part, in particular ground and/or polished, and the functional element 20 is additively constructed or manufactured on the surface of the femoral component 10. The additive construction or manufacture of the functional element 20 is preferably carried out by means of laser deposit welding, selective laser melting or selective laser sintering.

In respect of suitable materials for the base body, or the femoral component 10, and the functional element 20, reference is made to the materials disclosed in the general, or preceding, description for the base body and the at least one functional element.

The knee partial endoprosthesis 1 shown in FIG. 3 may also be referred to as a hybrid endoprosthesis in the context of the present invention.

FIG. 4 schematically shows a further embodiment of a medical product 1 according to the invention in the form of a knee partial endoprosthesis 1.

The knee partial endoprosthesis 1 comprises a base body in the form of a tibial component 10 and a functional element 20 connected, in particular connected by substance or material bonding, to the tibial component 10. The functional element 20 is preferably (likewise) configured in the form of a shaft.

In order to produce the knee partial endoprosthesis 1, the tibial component 10 is preferably used as a prefabricated component, that is to say as a prefab part, in particular ground and/or polished, and the functional element 20 is additively constructed or manufactured on the surface of the tibial component 10. The additive construction or manufacture of the functional element 20 is preferably carried out by means of laser deposit welding, selective laser melting or selective laser sintering.

In respect of suitable materials for the base body, or the tibial component 10, and the functional element 20, reference is made to the materials disclosed in the general, or preceding, description for the base body and the at least one functional element.

The knee partial endoprosthesis 1 shown in FIG. 4 may also be referred to as a hybrid endoprosthesis in the context of the present invention.

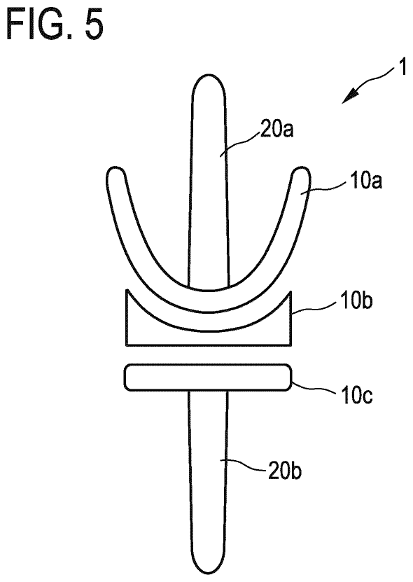

FIG. 5 schematically shows a further embodiment of a medical product 1 according to the invention in the form of a knee total endoprosthesis.

The knee total endoprosthesis 1 comprises a base body 10 and two functional elements 20a and 20b. Preferably, the two functional elements 20a and 20b are configured in the form of a shaft.

The base body 10 comprises a femoral component 10a, a tibial component 10c and a glide component or glide face component 10b arranged between them.

The functional element 20a is connected, preferably by substance bonding, to the femoral component 10a and the functional element 20b is connected, preferably (likewise) by substance bonding to the tibial component 10c.

In principle, the femoral component 10a and the tibial component 10c may be made of the same material or a different material.

In respect of suitable materials for the femoral component 10a and the tibial component 10c, reference is made to the materials disclosed in the general, or preceding, description for the base body.

Furthermore, in principle the functional elements 20a and 20b may be made of the same material or a different material.

In respect of suitable materials for the functional elements 20a and 20b, reference is made to the materials disclosed in the general, or preceding, description for the at least one functional element.

The glide component or glide face component 10b may for example be made from polyethylene, in particular ultrahigh molecular weight polyethylene.

In order to produce the knee total endoprosthesis 1, the base body 10 is preferably used as a prefabricated group of components, which comprises the femoral component 10a, the glide component or glide face component 10b and the tibial component 10c, or consists of these components, the functional element 20a is additively constructed or manufactured onto a surface of the femoral component 10a that faces toward the femur in the implanted state, and the functional element 20b is additively constructed or manufactured onto a surface of the tibial component 10c that faces toward the tibia in the implanted state. The additive construction or manufacture of the functional elements 20a, 20b is preferably carried out by means of laser deposit welding, selective laser melting or selective laser sintering.

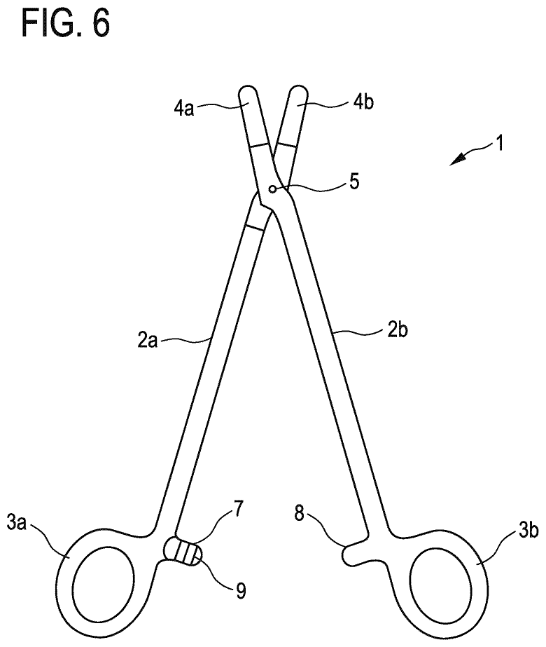

FIG. 6 schematically shows a further embodiment of a medical product 1 according to the invention in the form of a medical, in particular surgical, instrument.

The medical instrument 1 comprises two branches 2a, 2b, which are mounted rotatably or pivotably on one another in a link 5 or are connected to one another rotatably or pivotably. In this way, the branches 2a, 2b are movable relative to one another in rotation or pivoting, in particular shearing. On their proximal ends, the branches 2a, 2b each have a grip section 3a, 3b.

The link 5 may be configured as a bearing link or as a plug or box link.

The term “bearing link” is intended in the context of the present invention to mean a connection in which the two branches of a medical, in particular surgical, instrument are guided past one another, the branches each being flattened on a mutually facing side in an intersection region and a connecting element, for example in the form of a screw or a rivet, passing through the two branches and being anchored or fastened in the other branch. In the bearing link, one branch therefore lies on the other branch.

The term “plug or box link” is intended in the context of the present invention to mean a connection in which, in an intersection region of two branches of a medical, in particular surgical, instrument, one branch is flattened on both sides and the other branch has a through-hole. The branch flattened on both sides, which may also be referred to as a male branch, is guided through the branch comprising the through-hole, which may also be referred to as a female branch. In order to define a rotation or joint center, in this design a connecting element, for example in the form of a screw or a rivet, may also be guided through the two branches. Alternatively, for example, the male branch may comprise one or more, for example two, pins that are received in one or more, for example two, complementarily shaped pin sockets of the female branch.

The medical instrument 1 furthermore comprises two locking arms 7, 8. The locking arm 7 projects from the branch 2a in the direction of the branch 2b. The locking arm 8 projects from the branch 2b in the direction of the branch 2a, so that by means of engagement hooks 9 that are fitted on the locking arm 8 and engagement hooks (not represented) that are fitted on the locking arm 8, locking, in particular engagement, of the two locking arms 7, 8 and therefore of the medical instrument 1 is possible. The engagement hooks 9 of the locking arm 7 and the engagement hooks of the locking arm 8 are configured in such a way that form-fit locking is achieved when the grip sections 3a, 3b are pressed together. The engagement hooks may, for example, be configured in the form of shark teeth.

The branches 2a, 2b each comprise a functional section 4a, 4b at their distal ends. The functional sections 4a, 4b are each connected, preferably by substance bonding, to the branches 2a, 2b. The functional sections 4a, 4b may, for example, be configured as gripping or retaining jaws, holding and/or clamping jaws or cutting and/or separating jaws. Correspondingly, the medical instrument may for example be configured as a surgical gripping or retaining instrument, a surgical retention and/or clamp instrument or a surgical section and/or separation instrument in particular as surgical forceps, surgical tweezers, surgical needle holders or surgical scissors.

In order to produce the medical instrument 1, the branches 2a, 2b are used together with the link 5 and the grip sections 3a, 3b, preferably as a prefabricated group of components, and the functional sections 4a, 4b are additively constructed or manufactured on the distal ends of the branches 2a, 2b. The additive construction or manufacture of the functional sections 4a, 4b is preferably carried out by means of laser deposit welding, selective laser melting or selective laser sintering.

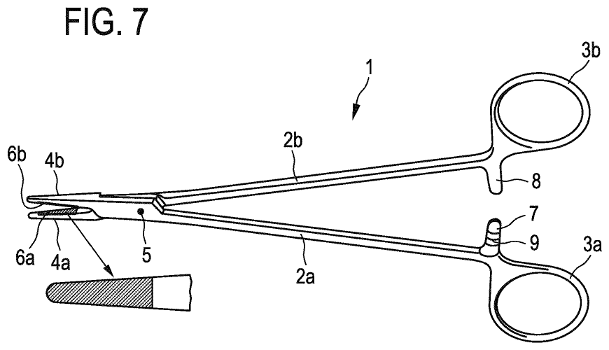

FIG. 7 schematically shows a further embodiment of a medical product 1 according to the invention in the form of a medical, in particular surgical, instrument.

The medical instrument 1 substantially corresponds in its structure to the medical instrument represented in FIG. 6, although the functional sections 4a, 4b each comprise an insert, in particular a metal insert, preferably a hard metal insert 6a, 6b.

In order to produce the medical instrument 1, the branches 2a, 2b are used with the link 5, the grip sections 3a, 3b and the functional sections 4a, 4b, preferably as a prefabricated group of components, and the inserts 6a, 6b are additively constructed or manufactured in recesses of the functional sections 4a, 4b. The additive construction or manufacture of the inserts 6a, 6b is preferably carried out by means of laser deposit welding, selective laser melting or selective laser sintering.

In respect of further features and advantages of the instrument 1 shown in FIG. 7, reference is made to the corresponding comments given in relation to FIG. 6, which apply accordingly. In particular, the references of FIG. 7 (apart from the references 6a, 6b, which have no counterpart in FIG. 6) have the same meaning as the corresponding references of FIG. 6.

1.-14. (canceled)

15. A method for producing a medical product, the method comprising the steps of:
a) manufacturing, or forming by additive construction, at least one functional element on a surface of a base body; and
b) connecting the at least one functional element to the base body after step a),
wherein the base body is produced by a non-additive manufacturing method.

16. The method according to claim 15, wherein step a) is carried out by deposit welding.

17. The method according to claim 15, wherein step a) is carried out by a beam sintering method or a beam melting method.

18. The method according to claim 15, wherein the at least one functional element is configured differently than the base body when carrying out step a).

19. The method according to claim 15, wherein a component, a component section or a group of components of an implant is used as the base body, and when carrying out step a) the component, the component section or the group of components of the implant is additively constructed or manufactured on the surface of the base body as the at least one functional element.

20. The method according to claim 19, wherein the implant is an endoprosthesis.

21. The method according to claim 20, wherein the endoprosthesis is selected from the group consisting of a hip joint endoprosthesis, a knee joint endoprosthesis, a shoulder joint endoprosthesis, an elbow joint endoprosthesis, an ankle joint endoprosthesis and a finger joint endoprosthesis.

22. The method according to claim 19, wherein the component or the group of components is selected from the group consisting of an artificial acetabulum, an artificial acetabular insert, an artificial femoral head insert, an artificial femoral head, a hip joint shaft, a cylinder or cone for a hip joint shaft, an upper leg prosthesis, a shaft for an upper leg prosthesis, a lower leg prosthesis, a shaft for a lower leg prosthesis, and groups of components consisting of at least two of the aforementioned components.

23. The method according to claim 15, wherein a component that is cylindrical or conical at least in sections is used as the base body, and when carrying out step a) a joint shaft is additively constructed or manufactured as the at least one functional element on a surface of the component.

24. The method according to claim 15, wherein an upper or lower leg prosthesis is used as the base body, and when carrying out step a) a shaft is additively constructed or manufactured as the at least one functional element on a surface of the upper or lower leg prosthesis.

25. The method according to claim 15, wherein a component, a component section or a group of components of a medical instrument is used as the base body, and when carrying out step a) the component, the component section or the group of components of the medical instrument is additively constructed or manufactured as the at least one functional element on the surface of the base body.

26. The method according to claim 25, wherein the medical instrument is selected from the group consisting of a dilation instrument, a grip instrument, a retention and/or clamp instrument, and a section and/or separation instrument.

27. The method according to claim 25, wherein the component, the component section or the group of components is selected from the group consisting of branches or branch sections, grips or grip sections, a link or link section, mouth parts or mouth sections, gripping or retaining jaws or gripping or retaining jaw sections, holding and/or clamping jaws or holding and/or clamping jaw sections, cutting and/or separating jaws or cutting and/or separating jaw sections, and groups of components consisting of at least two of the aforementioned components.

28. The method according to claim 15, wherein:
a group of components of a medical instrument is used as the base body, the group of components comprising two branches or branch sections mounted movably in rotation or pivoting on one another by a link, each branch or each branch section comprising a grip or grip section at a proximal end, and when carrying out step a) a functional section is additively constructed or manufactured as the at least one functional element on a distal end of the branch or of the branch section.

29. The method according to claim 15, wherein:
a group of components of a medical instrument is used as the base body, the group of components comprising two branches or branch sections mounted movably in rotation or pivoting on one another by a link, each branch or each branch section comprising a grip or grip section at a proximal end and a functional section at a distal end, each functional section having a recess for receiving an insert and when carrying out step a) an insert complementary in shape to a shape of the recesses respectively is additively constructed or manufactured as the at least one functional element in the recesses.

30. A medical product that is produced or producible by the method according to claim 15.

31. A medical product comprising:
at least one functional element produced by an additive or generative manufacturing method; and
a base body connected to the at least one functional element,
wherein the base body is not produced by an additive manufacturing method.
